(19) United States
(12) Patent Application Publication
(10) Pub. No.: US 2022/0176088 A1
(43) Pub. Date: Jun. 9, 2022

(54) SYSTEMS AND METHODS FOR MONITORING HEALTH CONDITIONS

(71) Applicant: Shifamed Holdings, LLC, Campbell, CA (US)

(72) Inventors: Amr Salahieh, Saratoga, CA (US); Tom Saul, Moss Beach, CA (US); Brian Fahey, Menlo Park, CA (US)

(21) Appl. No.: 17/599,354

(22) PCT Filed: Apr. 2, 2020
(86) PCT No.: PCT/US20/26301

§ 371 Date: Sep. 28, 2021

Related U.S. Application Data
(60) Provisional application No. 62/828,264, filed on Apr. 2, 2019.
(60) Provisional application No. 62/883,000, filed on Aug. 5, 2019.

(51) Int. Cl. A61M 27/00; A61M 39/22

(52) U.S. Cl. CPC A61M 27/002; A61M 39/22; A61M 2205/3334; A61M 2210/125; A61M 2205/3523; A61M 2205/52; A61M 2205/3306; A61M 2230/30

(57) ABSTRACT

The present technology relates to interatrial shunting systems and methods. In some embodiments, the present technology includes a system for shunting blood between a left atrium and a right atrium of a patient. The system can include a shunt having a lumen extending therethrough. When the shunt is implanted in the patient, the lumen is configured to fluidly couple the left atrium and the right atrium. The system can also include a sensor configured to be implanted in the patient and operably coupled to the shunt. The sensor can be configured to measure one or more parameters corresponding to a physiological parameter of the patient and/or a characteristic of the shunt. The system can further include an external component wirelessly coupled to the sensor. The external component can be worn by or otherwise adhered to the patient.

### CROSS-REFERENCE TO RELATED APPLICATION(S)

This application claims priority to U.S. Provisional Patent Application No. 62/828,264, filed Apr. 2, 2019, and U.S. Provisional Patent Application No. 62/883,000, filed Aug. 5, 2019, the disclosures of which are incorporated by reference herein in their entireties.

### INCORPORATION BY REFERENCE

All publications and patent applications mentioned in this specification are herein incorporated by reference to the same extent as if each individual publication or patent application was specifically and individually indicated to be incorporated by reference.

### TECHNICAL FIELD

The present technology generally relates to monitoring a patient's health and, in particular, to systems for monitoring a patient's health having both a wearable component and an implantable component.

### BACKGROUND

In the early stages of heart failure, compensatory mechanisms occur in response to the heart's inability to pump a sufficient amount of blood. One compensatory response is an increase in filling pressure of the heart. The increased filling pressure increases the volume of blood in the heart, allowing the heart to more efficiently eject a larger volume of blood during each heartbeat. Increased filling pressure and other compensatory mechanisms can initially occur without overt heart failure symptoms.

The mechanisms that initially compensate for insufficient cardiac output, however, lead to heart failure decompensation as the heart continues to weaken. The weakened heart can no longer pump effectively, which can cause increased filling pressure and lead to chest congestion (thoracic edema) and heart dilation, which further compromises the heart's pumping function. This cycle of heart failure generally leads to hospitalization.

Typically, therapy for a patient hospitalized for acute decompensated heart failure (ADHF) includes early introduction of intravenous infusion of diuretics or vasodilators to clear fluid retained by the patient. This therapy can be highly effective in reducing ADHF symptoms rapidly, but overdiuresis can occur if the intravenous infusion of drugs is delivered too long or at too high of a dosage. Since there is a lag in time between reaching an optimal fluid volume status and the alleviation of symptoms, determining the optimal parameters for controlling the intravenous infusion therapy remains a challenge to clinicians. Overdiuresis may require fluid to be administered to the patient to increase the patient's fluid volume status. Removing and adding fluid can pose additional burden on the kidneys, which may already be compromised due to renal insufficiency in the heart failure patient. At other times, the fluid removed may not be sufficient to achieve a desired result.

For 2011 to 2012, the annual total costs of cardiovascular disease in the United States were estimated to be $316.6 billion. Factors that continue to drive the prevalence of cardiovascular disease include advanced age and rising rates of obesity, diabetes, and heart-attack survival. Despite recent advances in medical treatment options, heart failure remains a leading cause of hospitalization in people over the age of 65. In some patients, chronic stable heart failure may easily decompensate, resulting in patient hospitalization or even mortality. Recurrent hospitalizations stemming from ADHF events result in significant patient mortality and health-care costs.

Heart failure includes a spectrum of conditions that prevent the heart from supplying enough blood to meet the body's oxygen demands. Heart failure affects almost 6 million people in the US, and more than 20 million people worldwide. It is generally not a curable disease, but it can often be controlled. Patients whose heart failure is controlled (e.g., their bodies are compensating for their heart's inability to circulate enough blood, usually through an increase in the heart's overall effort) frequently lapse into acute episodes of decompensated heart failure (DHF), in which their bodies are no longer able to compensate for their hearts' shortcomings. DHF commonly results in hospitalization. Patients who have been recently hospitalized for heart failure (e.g., those who generally leave the hospital in a compensated state) are at particularly high risk for decompensation. Consequently, about 25% of heart failure patients who are discharged from a hospitalization are re-admitted to a hospital within 30 days.

Consequently, there is an increased focus on identifying decompensation in progress before hospitalization is required. In this way, patients can be managed through phone-based interventions or outpatient clinic visits, which cost far less than hospitalization and are far less disruptive to patients' lives. Three approaches are commonly used to assess a patients' risk of decompensation and readmission to the hospital: (1) static risk estimation, (2) human telemonitoring, and (3) invasive signal thresholding.

In the first approach, hospitals use a dashboard that identifies patients at high risk at the time of discharge, who are then targeted for subsequent follow-up. This is a simple estimation approach based on a static “snapshot” of the patient's clinical data, and is unable to adapt to the myriad changes that take place after a patient is discharged.

In the second approach, hospitals employ a team of clinicians who are responsible for contacting recently discharged heart failure patients to ask them about their activity levels and symptoms (in particular weight and shortness of breath), and to make remote treatment adjustments for patients who appear to be getting worse. This process is expensive and requires significant time from caregivers. Interpreting data obtained in this manner is highly subjective, and decompensation may only be identified after it has progressed far enough to manifest as symptom changes that are apparent to the patient. In many cases, it is thought that this occurs too late for optimal or effective intervention.

The third approach depends on implanted devices, for example, either thoracic fluid monitors that are implanted in or near the heart, or pressure monitors that are implanted in the pulmonary artery. In general, these devices can be configured to generate alerts when the measured quantities exceed simple thresholds. These classes of devices are expensive, highly invasive (and thus only utilizable by certain patients), and measure only a single quantity that is compared to an empirical threshold.

Each of these approaches to risk prediction for decompensation in heart failure patients focuses on simple models based on static aspects of the patient (metrics known at the time of hospital admission, discharge, or other clinical visit) and/or on thresholding infrequently collected measurements (weight, symptom reports), or a single continuously collected measurement (pulmonary artery pressure, thoracic congestion).

There is a need to obtain more types of patient data over time to be able to better monitor and understand the patient's condition as is relates to heart failure. There are also needs to more accurately assess patient compliance and increase patient compliance with one or more heart failure treatment plans.

### BRIEF DESCRIPTION OF THE DRAWINGS

FIGS. 1A and 1B illustrate components in an exemplary traditional system, and a block diagram of such a traditional system.

FIG. 2A illustrates a patient interface and positioning of a sensor configured in accordance with select embodiments of the present technology.

FIG. 2B illustrates a block diagram of a system, including the patient interface from FIG. 2A, configured in accordance with select embodiments of the present technology.

FIG. 3 illustrates an embodiment of a system, including placement of an intraarterial pressure sensor, configured in accordance with select embodiments of the present technology.

FIG. 4 illustrates a patient interface, including a position of a pressure transducer, configured in accordance with select embodiments of the present technology.

FIG. 5 illustrates a distal end of the access device of FIG. 3 and an introducer configured in accordance with select embodiments of the present technology.

FIGS. 6A, 6B, 6C, and 6D illustrate a patient interface configured in accordance with select embodiments of the present technology.

FIG. 7 illustrates another patient interface configured in accordance with select embodiments of the present technology.

FIG. 8 illustrates a wearable device and a partially implantable device configured in accordance with select embodiments of the present technology.

FIG. 9 illustrates a wearable device and a partially implantable device configured in accordance with select embodiments of the present technology.

FIG. 10 illustrates aspects of a partially implantable device configured in accordance with select embodiments of the present technology.

FIG. 11 illustrates aspects of a partially implantable device configured in accordance with select embodiments of the present technology.

FIGS. 12A and 12B illustrate an applicator tool for deploying a wearable device over an implanted component and configured in accordance with select embodiments of the present technology.

FIG. 13 is a graphical illustration of the absorption coefficient for various forms of hemoglobin at various wavelengths.

FIG. 14 is a graphical illustration of the absorption coefficient for plasma at various wavelengths.

FIG. 15 is a graphical illustration of pulsatile and non-pulsatile components that affect the amount of fluid in a volume of tissue at any given time.

FIGS. 16A and 16B illustrate a system for measuring the amount of fluid in a volume a tissue that includes an implantable component and an external component and is configured in accordance with select embodiments of the present technology.

FIGS. 17A-17C illustrate a system for monitoring and/or treating heart failure in a patient and configured in accordance with select embodiments of the present technology.

### DETAILED DESCRIPTION

The present technology is generally related to systems, devices, and methods for treating, monitoring, diagnosing, or otherwise addressing heart failure. For example, some embodiments of the present technology relate to interatrial shunting systems and methods. For example, in some embodiments the present technology provides a system for shunting blood between a left atrium and a right atrium of a patient. The system can include a shunt having a lumen extending therethrough. When the shunt is implanted in the patient, the lumen is configured to fluidly couple the left atrium and the right atrium. The system can also include a sensor configured to be implanted in the patient and operably coupled to the shunt. The sensor can be configured to measure one or more parameters corresponding to a physiological parameter of the patient and/or a characteristic of the shunt. The system can further include an external component wirelessly coupled to the sensor. The external component can be worn by or otherwise adhered to the patient (e.g., as a “wearable” device).

In some embodiments, the present technology provides systems and methods for monitoring compliance with and/or increasing compliance of patients with a treatment for heart failure. Some aspects may include providing guidance for modifying at least one aspect of a patient's behavior related to increasing compliance with a heart failure therapy and/or treatment plan. Heart failure is a serious condition that can often be deadly. Accordingly, patients and caregivers have a strong incentive to follow directions to lengthen lifespan and/or decrease suffering.

Some aspects of this disclosure may be directed to diagnostics related to heart failure. For example, some aspects of the disclosure are related to communicating patient data and/or information related to heart failure to one or more devices and/or systems so that one or more individuals (e.g., a nurse, physician, patient, etc.) can gain access to the data and/or information.

Increased compliance with best standard practices and treatments for addressing heart failure can greatly improves a patient's health. Accordingly, as provided above, some aspects of the disclosure are related to increasing compliance with a heart failure treatment plan. For example, some aspects of the disclosure are related to increasing compliance with one or more of the following activities of a treatment plan: taking one or more drugs prescribed by a physician(s) and following a proper schedule; taking/obtaining (which may occur automatically and without requiring patient involvement) and sending to medical personnel (manually or automatically) daily measurements such as weight, blood pressure, glucose, pulse rate, ECG, oxygen saturation, and/or any other measurement described herein, as well as changes in and/or rates of change in any of the foregoing measurements; maintaining a certain diet; attending and/or scheduling physician visits; activity in peer group support (e.g., online or in-person); maintaining strong social ties, etc. Aspects of this disclosure may reduce the costs for care providers by one or more of the following: improving patient monitoring (e.g. improving the amount of patient data being obtained), reducing patient hospitalizations, and/or reducing the cost of monitoring by flagging non-compliant patients and focusing costs and efforts on needy patients.

The disclosure herein sets forth a variety of devices and systems that can be used to perform or accomplish one or more of the functions herein. The devices and systems are exemplary and are not necessarily limiting. The devices may include aspects, elements, or features that are not necessarily needed to accomplish or perform all functions herein. Suitable device and/or system features from different embodiments and figures can be combined unless indicated herein to the contrary.

An advantage of some of the devices, systems, and methods herein is that many different types of patient information can be obtained. Having access to additional types of patient information allows greater cross correlation between the different types of information, which can provide enhanced insight into and greater accuracy about the status of one or more patient conditions, such as the status of a patient's heart failure.

Some aspects of this disclosure include one or more wearable devices. Some aspects that include one or more wearables also include one or more portions of a system that are adapted to be (and/or are) implanted in a patient. Implantable in this context refers to a device in which at least a portion is disposed below the external surface of a patient's skin. This can include, for example, devices that are fully implanted (e.g., an interatrial shunt device, “IASD”) as well as devices that include an external (e.g., non-implanted) component and an internal (e.g., implanted) component operably coupled to the external component.

One manner in which many different types of patient information can be obtained is by incorporating a plurality of sensors into a wearable device. In addition to sensors incorporated into a wearable device, one or more sensors (at least a portion of a sensor) may be disposed below an external surface of a patient's skin. Patient information can then be obtained using the sensors, where the information from each sensor individually and/or and the sensors as a group is related to one aspect of a patient's heart failure. The sensed information, either raw or processed to some extent, can be used in a variety of ways to provide one or more indicators of the patient's health. By having more data points based on the different types of sensed patient information, more accurate estimates and/or determinations can be made about the patient's health.

Therefore, some aspects of the disclosure and embodiments herein include a wearable device that is, when in use, positioned adjacent to the skin of a patient and in relatively close proximity to a region of the patient that is used to obtain information and/or data. In some instances, for example, it may be helpful to have one or more wearable devices in a fixed position relative to the skin and/or the region of the patient.

In some embodiments, a system may include a wearable device intended to be reused and a second patient device. The second patient device can be adapted so that a portion is adapted and sized to be positioned/implanted (including temporarily implanted) under an external surface of the skin (e.g., intravascular pressure sensor, IASD, etc.), and optionally a second portion is adapted to be placed on the external surface of the skin (e.g., a patch), and if the second patient device includes an external portion, the internal portion and the external portion may be in wired or wireless connection. In these embodiments, the “second patient device” may be referred to herein as an “implantable device” even if part of the device is disposed outside the patient. An external portion of the second patient device may also be considered a “wearable” or wearable portion in that it may be worn by the patient (e.g., a patch portion of the second patient device/implantable device). The second device may in some uses be disposable with a useful life of a few days to several months, such as three months.

In some embodiments herein the implantable device may include one or more features that helps improve the efficiency and or quality of the data sensing/gathering using one or more sensors in the one or more wearable devices. Typical efficiency and quality improvements can include more frequent readings, higher frequency readings, improved signal to noise, to name a few.

The disclosure herein, including any specific embodiments below, may be related to the disclosure in PCT Publication No. WO2017165879, which is incorporated by reference herein for all purposes. For example, any of the devices, systems and methods of use related to sensing blood pressure described in WO2017165879 may be incorporated into any of the embodiments herein unless indicated to the contrary herein.

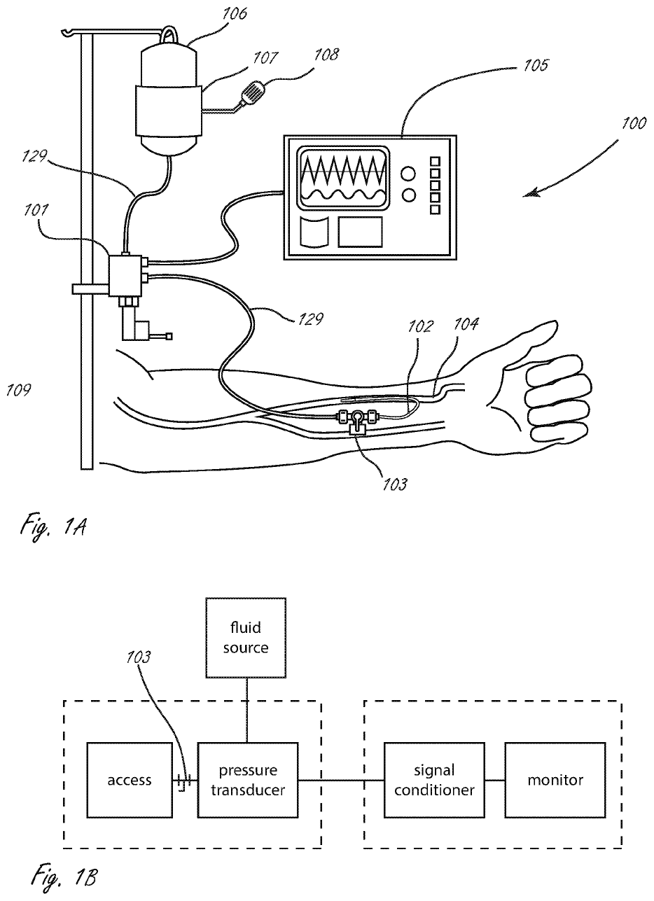

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to FIGS. 1-17C.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

FIG. 1A illustrates the primary components of an exemplary traditional intravascular blood pressure monitoring system 100 configured for monitoring arterial pressure. The patient interface comprises an access device 102 partially positioned in a blood vessel 104 (e.g., a radial artery as shown), a valve (e.g., stopcock) 103, a pressure transducer 101, and a tubing set comprising a fluidic path 129 for fluidically coupling the described components. The system 100 also comprises a fluid reservoir 106 of sterile fluid (e.g., saline) to constantly flush the transducer 101 and associated lines, a pressurizing device 108 (e.g., a pressure bulb) adapted to apply pressure to the fluid reservoir 106 with pressure cuff 107, associated tubing to connect the fluid reservoir 106 to the pressure transducer 101, and an instrument or monitor 105 to power and condition the signal from the pressure transducer 101 and other transducers, and/or monitor, record, and/or present a representation of the signals detected by the transducers. The fluid is used to flush the transducer 101 and thereby prevent blood from clotting within the fluidic path, which may cause the pressure detected at the pressure transducer 101 to not be reflective of the actual physiological pressure. Such a system can also be adapted to access arterial blood for the purposes of collecting blood samples when they are required on a relatively frequent basis, such as when blood gas values are required. FIG. 1B illustrates a block diagram representation of such a system from FIG. 1A.

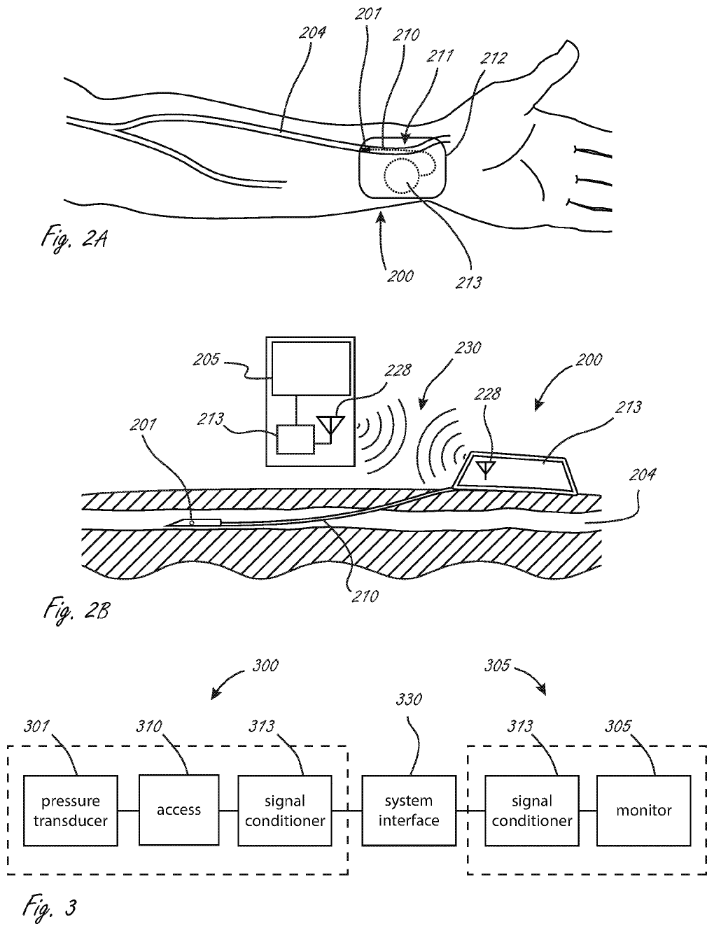

FIGS. 2A and 2B illustrate various views and/or aspects of some embodiments of an ambulatory radiofrequency link enabled intravascular monitor (ARLIM) system. The illustrated embodiment in FIGS. 2A and 2B includes a patient interface 200 and a monitor 205. The patient interface 200 comprises an indwelling portion 211 and an external portion 212. The indwelling portion 211 comprises an access device 210 and a pressure transducer 201. The external portion 212 comprises signal conditioning components 213 and a system interface 230 facilitated by radiofrequency (RF) transceivers 228 adapted to communicate between the monitor 205 and a patient interface 200. The monitor 205 is configured for monitoring the data stream provided by the patient interface, processing the data, sending the data to another user, and/or the like.

In particular, FIGS. 2A and 2B illustrate the access device 210 positioned in a radial artery 204. As illustrated, the pressure transducer 201 is disposed on a distal region (in this embodiment the distal end) of the access device 210. In other embodiments, the access device 210 provides a transmission path for pressure pulses to a pressure transducer disposed in the external portion of the access device 210.

FIG. 3 is a block diagram illustrating one embodiment of a patient interface 300 and monitor 305. In the illustrated embodiment, the pressure transducer 301 is disposed at the distal end of the access device 310 of the indwelling portion of the patient interface 300. The patient interface signal conditioner 313 is powered and can include an RF transceiver. Likewise, the signal conditioner 313 in the monitor 305 can include an RF transceiver. The system interface 330 can be an RF link, such as a Bluetooth or other suitable link. In such an embodiment, information may be transmitted from the patient interface 300 to the monitor 305, such as, without limitation, calibration factors, changes in data collection rate, or characteristics of the pressure waveform, data precision.

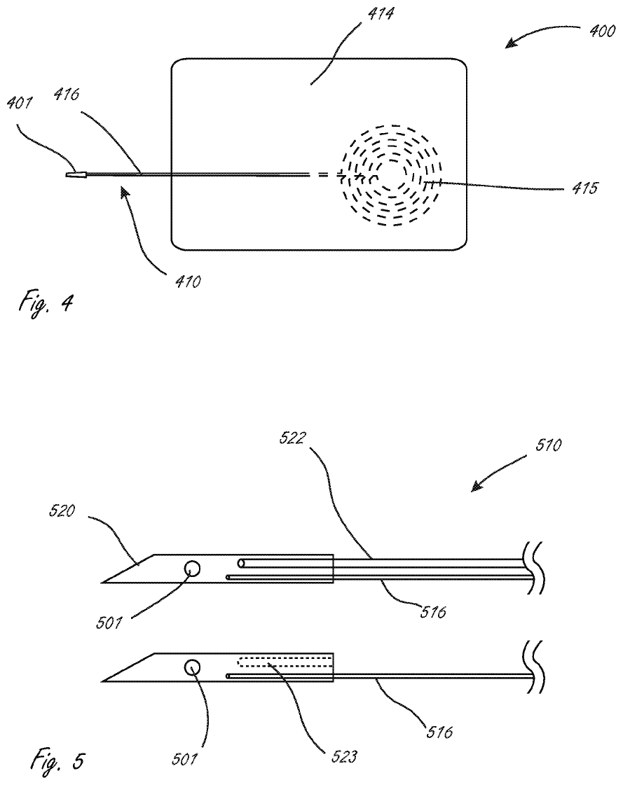

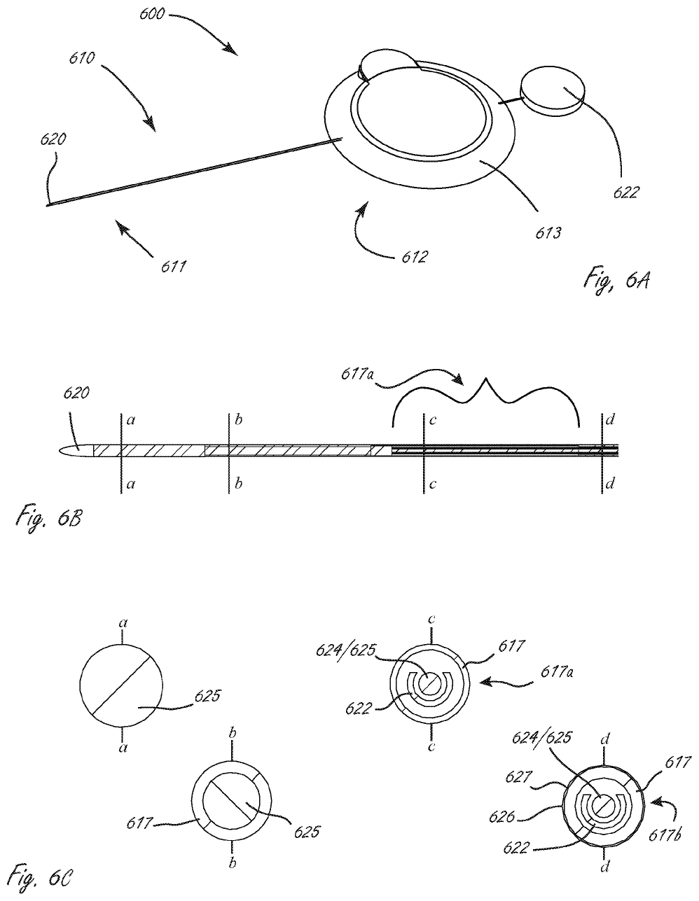

FIG. 4 illustrates an embodiment of the system shown generally in FIGS. 2A and 2B, wherein the patient interface 400 comprises a pressure transducer 401 and a passive signal conditioner 414. As illustrated, the signal conditioner 414 comprises an antenna 415 and additional circuit elements (not shown). In such a device, changes in the transducer characteristic associated with changing pressure causes a change in a characteristic as measured by the system interface (not shown). This change can be expressed as a change in impedance and/or resonant frequency of the patient interface. In such a system power can be derived from the RF link.

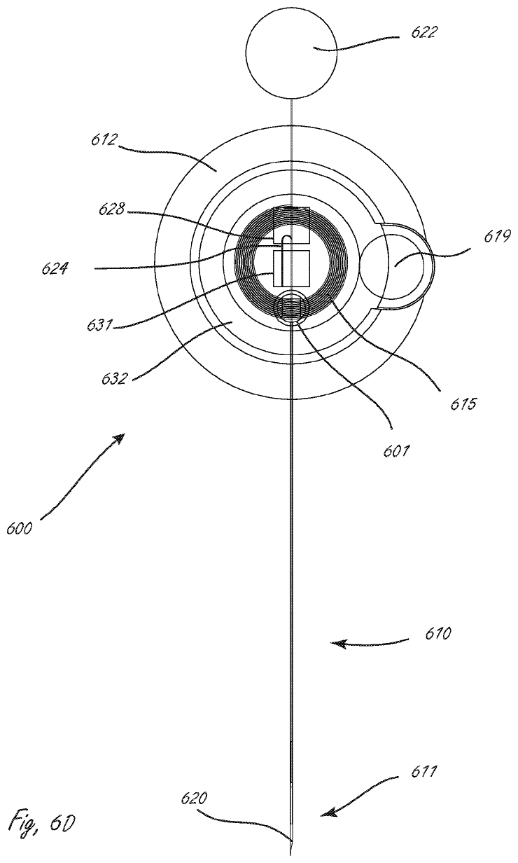

FIG. 5 illustrates a distal region of an access device 510 configured in accordance with an embodiment of the present technology. The device 510 comprises a pressure transducer 501 disposed near the distal region. The distal region is configured with a sharpened end, or sharps component 520, for insertion through the artery wall. The sharps component 520 is coupled to the signal conditioning component (not shown) via an electrical interface such as a cable or flexible circuit 516. To provide the access device 510 with sufficient rigidity to deliver the pressure transducer into the artery, the sharps component 520 includes an introducer interface 523 adapted to be releasably interfaced with an introducer, such as a stylet 522. Once delivered, the stylet 522 may be removed, thereby providing a very small cross section and very flexible indwelling access component, which minimizes trauma to the patient. In some embodiments the introducer (e.g., the stylet 522) and sharps component 520 can also be non-releasably secured. The sharps component 520 may also be referred to herein as a sharpened distal housing, which may be made of multiple components secured together to form the housing rather than a single housing structure made from a single starting material.

FIG. 6A illustrates features of a patient interface device 600 configured in accordance with the present technology. FIG. 6B is a side view of a distal region of the indwelling portion 611, and shows four cross sections. Referring to FIGS. 6A and 6B together, the patient interface 600 includes an access device 610 comprising an indwelling portion 611 and an external portion 612 for coupling pressure pulses to the signal conditioning unit 613, and an introducer, such as a delivery stylet 622, configured to stiffen access components structurally during the delivery process. The stylet 622 passes through signal conditioning unit 613 during delivery of the access component and is removed after delivery.

FIG. 6C shows four cross sections taken from FIG. 6B. The distal end of the access device, which is distal to section a-a, comprises a sharpened end 620 and can be a solid section, such as a solid stainless steel section. Section b-b shows an interface between an outer, elastomeric, non-covered, section of catheter 617, and the inner structure 625 that comprises a one-way valve, allowing fluids delivered from the catheter to be released into the bloodstream and/or the fluid coupling region to be purged and or filled (as described above). The section c-c is within the catheter blood pressure capturing zone, and includes a thin walled section 617a of the catheter 617, and a reduced cross section inner member or wire portion 624 that acts as a tether to prevent losing the distal end region and interface with the stylet during delivery. Section d-d is within the pressure transmitting zone and comprises the tether 624 or other elongate device, and a thicker walled 617b portion of the catheter 617. This section of the catheter may, in addition, be covered with a non-elastomeric polymer such as a poly ethylene or fluoropolymer to add additional stiffness to the catheter. The optionally removable stylet 622 is shown in sections c-c and d-d, configured to slidably interface with the tether or other elongate device. In alternative embodiments, only a portion of the wall in 6C(c) is thinner (i.e., less than 360 degrees), such as half of the wall, or a quarter of the wall.

FIG. 6D illustrates the patient interface 600 with some components contained within the signal conditioning unit 613 comprised within the external portion 612 made visible. As illustrated, a battery 619 is used to power the signal conditioning unit 613 comprising pressure transducer 601 and conditioning circuitry 631. Conditioning circuitry 631 converts the pressure signal into a radio signal compatible with being broadcast by the RF transceiver 628 that interfaces with antenna 615. Typically, the pressure signal will be converted to a digital format and passed to the RF transceiver 628. Also shown is the proximal termination of the tether 624 anchored in the body of the signal conditioner housing 632. Without the stylet, only the distal region of the indwelling portion has sufficient stiffness to be advanced into and through the vessel. The stylet, when inserted, extends along more than 50% of the indwelling portion (and in this embodiment more than 75%), and allows the much more flexible proximal section to be advanced through the vessel.

In the illustrated embodiment, the pressure transducer is housed in the external portion of the patient interface. The pressure transducer 601 is operably coupled to blood pressure via a pressure coupling fluid (e.g. liquid) or gel within sealed access device 610. The device is thus adapted and configured to transmit pressure pulses from the blood to the pressure transducer. Referring back to FIG. 6B, the access device 610 comprises a tubular outer member or catheter 617 typically constructed of an elastomeric material (silicone, poly urethane PEBAX, or other such material, or copolymers of such materials), and in some embodiments, covered in areas with a thin, more rigid polymeric material (FEP, PTFE, PE, or other such materials or copolymers of such materials). As best illustrated in FIG. 6C, the access device 610 can also include an inner structure 625, and a removable delivery stylet 622. The catheter 617 interfaces at its distal end with a distal section of the inner structure 625 in an interference fit, as can be seen in FIG. 6B, and in the cross section b-b shown in FIG. 6C. As shown, the catheter 617 is disposed around inner structure 625 in an interference fit with inner structure 625. The most distal portion of the inner structure 625 comprises a sharpened end 620 to allow insertion of the access device into the vessel through tissues.

The distal region of the indwelling portion of the catheter is adapted to act as a one-way valve, which allows coupling media delivered under pressure from within the access device to be delivered to the blood, but does not allow blood to enter the inside of the catheter. As such, the blood is isolated from the pressure transmitting media but the fluid-pressure coupling region may be purged and/or filled with the pressure coupling media. In this particular embodiment, and in reference to section b-b, the tubular member 617 is an elastomeric material, while the inner member 625 is a much stiffer material such as (for example) stainless steel or a relatively hard plastic. When pressure transmitting media is delivered under pressure into the access device 610, the elastomeric material of tubular member 617 will distend slightly (relative to the inner member 625) in response to the increase in fluid pressure, which will allow fluid to pass out of the catheter and into the blood. When the pressure transmitting media is no longer delivered, the pressure from the blood external to the catheter will maintain the interference fit between the tubular member 617 and the inner member 625, thus preventing blood from entering the indwelling portion. In this manner, the pressure coupling volume within the indwelling portion can be purged of air, and optionally fluids may be delivered to the blood stream.

Pressure pulses are transmitted into the pressure transmitting media via a relatively thin-walled section 617a of the catheter portion of the access device, which is compliant enough to allow pressure pulses to be transmitted from the blood into the coupling pressure media. The blood is thus in pressure communication with the pressure transducer, but the blood is not in direct physical contact with the media inside the catheter. In alternate embodiments the section 617a may encompass less than 360 degrees of the tubular member.

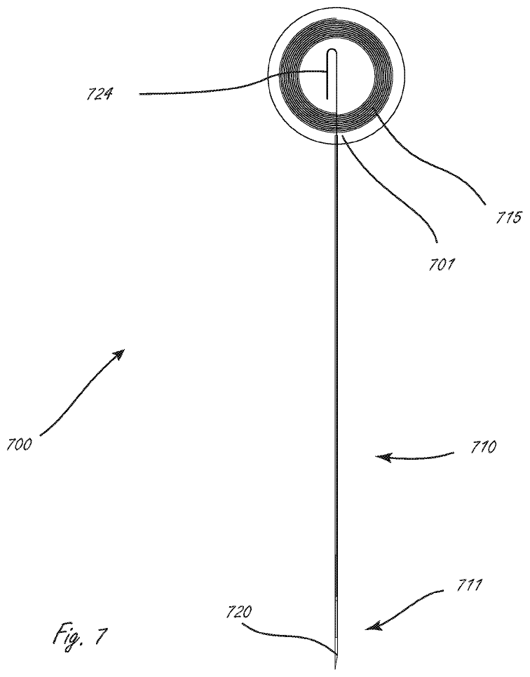

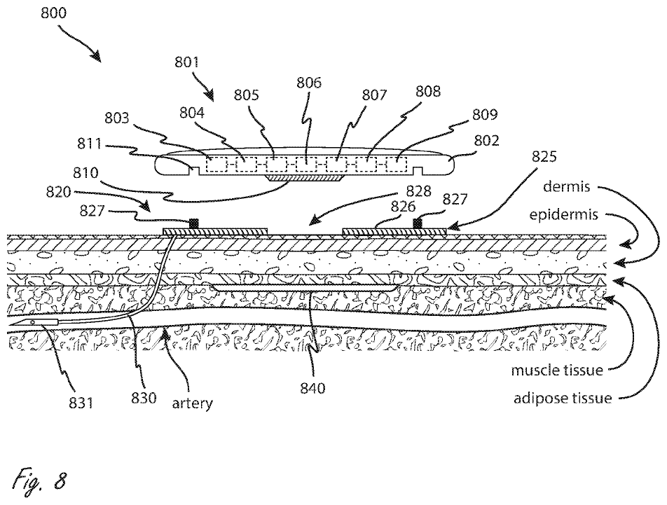

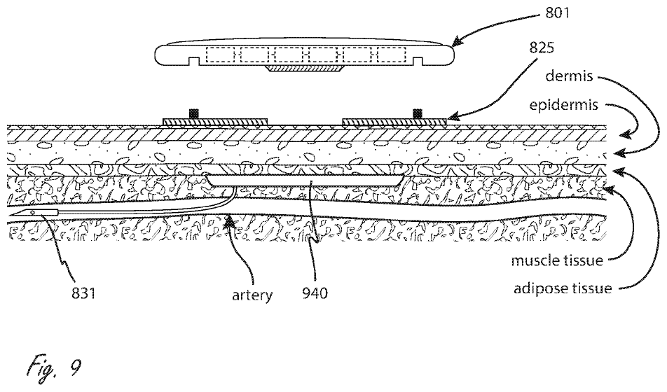

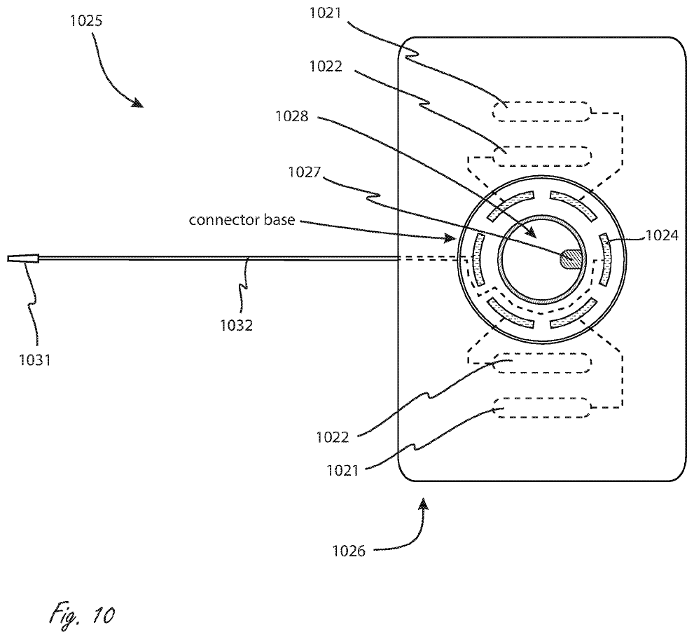

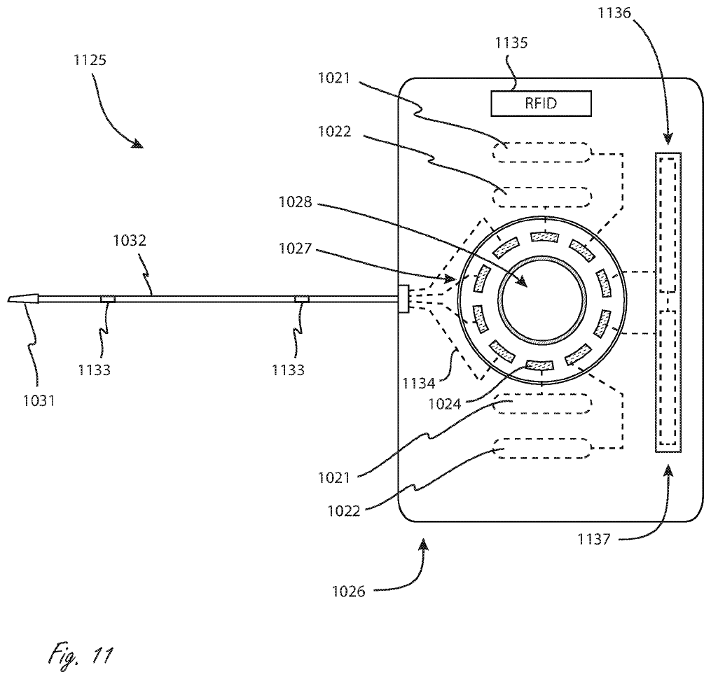

FIG. 7 illustrates a patient interface 700 that can be generally similar in some aspects to the patient interface 600 described with respect to FIG. 6D. The patient interface 700 can include, for example, an access device 710 having an indwelling portion 711 with a sharpened end 720. The patient interface 700 can also include a pressure transducer 701 and an antenna 715. The patient interface 700 can further include a wire portion 724 that acts as a tether to prevent losing the distal end region and interface with a stylet during delivery, as described with respect to FIG. 6D.

FIG. 8 illustrates a system 800 configured to detect or sense one or more patient signals that can be indicative of patient information, optionally related to the status of a heart failure condition and/or status of compliance with a heart failure treatment plan. FIG. 8 also illustrates an exemplary location for placement of the system 800, including the different components. “Signals” as used herein includes any type of information sensed by one or more sensors (e.g., optical, pressure, electrical, etc.). The exemplary system 800 in FIG. 8 includes a wearable 801, as well as a second patient device 820 that includes a surface portion 825 (e.g., a docking station for the wearable 801) of the implant and an implantable portion 830 that in this embodiment includes an implanted sensor 831 (e.g., a pressure sensor). In this embodiment the surface portion 825 of the implant is coupled to the implantable portion 830, but in alternative embodiments this need not be the case (e.g., FIG. 9). As described in greater detail below, the surface portion 825 can include a patch 826 having one or more contact posts for receiving the wearable 801. The surface portion 825 can also include an optical port 828 extending through the patch 826. In some embodiments, the surface portion 825 is omitted and the wearable 801 can interface directly with the skin.

In FIG. 8, the wearable 801 includes a housing 802 (which may be comprised of one or more components secured together), which supports a plurality of components. This exemplary wearable includes a power source 803 (rechargeable or not), one or more pressure sensing components 804 (e.g., a pressure sensor), one or more motion components 805 (e.g., an accelerometer), one or more ultrasound components 806 (e.g., an ultrasound transducer), one or more optical components 807 (e.g., optical emitters (e.g., LED(s) and/or optical sensors), one or more impedance components 808 (e.g., electrodes for measuring tissue impedance signals), and a radio link 809 to communicate data with one or more other devices. In this embodiment the wearable device 801 also includes an optical sensor 810 configured and positioned to sense light, which may be reflected light emitted from a light source (e.g., one or more LEDs in the one or more optical components). The wearable 801 can be configured so that the power source 803 provides power to one or more of the components. The wearable 801 need not include all of the components and sensors shown. In some alternatives the wearable can include any combination of components shown in FIG. 8 or any other embodiment herein. The locations of the different components in FIG. 8 is not necessarily indicative of where they must be in the wearable 801, but is rather an illustration showing the different exemplary components and the general exemplary form factor of a wearable.

The wearable 801 also includes one or more implant interface features 811. In this embodiment the one or more implant interface features 811 can physically interface with one or more interface features on a patch (e.g., the surface portion of the implant) to help stabilize the position of the wearable 801 relative to the surface portion 825. Controlling the position of the wearable 801 relative to the surface portion 825 can also control the position of the wearable 801 relative to an implantable portion 830. This can help control the relative positions of an external component and an internal component, where there is an optimal relative positioning of the external and internal components (e.g., positions of a light emitter and a light sensor relative to an internal reflector).

In this embodiment the interface between the wearable 801 and the patch portion 825 also creates an electrical connection between the wearable 801 and the surface portion 825, thus allowing for electrical communication between the wearable 801 and the secondary device 820. For example, power can be supplied from the wearable 801 to the surface portion 825 and/or implanted sensor(s) 831. Additionally, signals can be communicated from the surface portion 825 to the wearable 801 via the contacts/interface (e.g., signals indicative of blood pressure sensed by the implanted sensor 831).

As provided above, the wearable 801 also includes the optical sensor 810, which has a surface extending away from a bottom surface of the wearable housing 802 so that it extends further away from a housing bottom surface. This protruding configuration allows the optical sensor 810 to interact with the optical port 828 in the external portion 825. By having the optical sensor 810 disposed in a region of the surface portion 825 that does not include the patch material, light signals do not have to pass through the patch 826 to reach the optical sensor 810, providing less interference with optical transmission.

The system 800 can further include an additional implantable component such as a “reflector or sensor” 840. The reflector/sensor 840 can be a subcutaneously positioned device (as shown) adapted to, for example, act as a light reflector and/or a light sensor that can be adapted to reflect and/or sense light emitted from one or more light emitters in the wearable, as is described elsewhere herein. For example, the implanted reflector/sensor 840 can allow the system 800 to work in single path length transmissive mode (e.g., as a sensor) or dual pathlength transmissive mode (e.g., acting as a reflector), or configured as both, with one or more portions configured as a reflector and one or more portions acting as a sensor. Such a system would allow for better SSNR in areas where the skin is relatively thin and blood volume in the capillary bed is minimal.

FIG. 8 also illustrates an implanted sensor 831 that is indirectly physically coupled to the surface portion 825 of the implant, such as any of the implanted blood pressure devices herein. The implanted “sensor” 831 may also be a device or assembly that does not include a typical “sensor” per se but is adapted and configured to communicate information indicative of a patient parameter, such as blood pressure. The implanted sensor 831 can be a variety of known intravascular pressure sensors (e.g., the CardioMEMS pulmonary artery pressure sensor) modified to be in physical connection with the surface portion 825 of the implant 820.

FIG. 9 illustrates a system 900 configured in accordance with another embodiment of the present technology. The system 900 is generally similar to system 800 described above with reference to FIG. 8, except that the implanted sensor 831 is not physically coupled to the surface portion 825. Instead, in system 900, the implanted sensor 831 is indirectly physically coupled to a sensor assembly 940. The sensor assembly 940 can include an antenna (not shown) to communicate information detected from the implanted sensor 831 to the surface portion 825 of the implant and/or the wearable. Alternatively, communication may occur using ultrasound and/or an optical mechanism. For example, an optical communication can include LCD absorbance. The internal component may be configured to use relatively low power to communicate, while the external wearable, due to its larger size, may be configured with a higher power source that can enable it to use higher power. All other aspects of the system from FIG. 8 can be incorporated by reference into this embodiment.

FIGS. 10 and 11 provide top views of alternative embodiments having some features in common with the systems illustrated in FIGS. 8 and 9. More specifically, FIGS. 10 and 11 illustrate a surface portion 1025 physically coupled indirectly with an implanted sensor 1031 (e.g., a pressure sensor) via a sensor lead 1032. The surface portion 1025 can in some embodiments be referred to as a “wearable portion,” and in some ways can be similar to the wearables and/or the surface portions of the implantable device shown in FIGS. 8 and 9. The surface portion 1025 in the embodiments in both FIGS. 10 and 11 include electrode exciter pairs 1021 and electrode reader pairs 1022, which are adapted to monitor electrical impedance across adjacent tissue. Electrical connections from the exciters 1021 and readers 1022 are shown in dashed lines, as are leads from the pressure sensor 1031. The arc regions 1024 in the surface portion 1025 are electrical regions that can be placed into electrical contact with portions of another device (e.g., the wearable 801 when the wearable 801 is coupled to the surface portion 1025) to communicate data/information from and/or to individual electrical components. The wearable can include any of the sensors shown in FIGS. 8 and 9. In some alternatives a cable can replace and extend from the wearable the individual electrical contacts that are shows as arcs of a circle and the cable extends to a connector on the wearable. The surface portion 1025 can also include a connector 1027 for receiving another device (e.g., the wearable 801 of FIG. 8).

FIG. 11 illustrates a surface portion 1125 and coupled pressure sensor 1031, with additional components shown compared to the arrangement shown in FIG. 10. The implanted portion has a shaft that is carrying one or more ECG sensors 1133 (two shown) that can record electrical activity, which can be communicated via ECG leads 1134 (four leads are shown extending from the shaft). This embodiment also includes a RFID tag 1135 embedded in the patch 1026, which allows the surface portion 1125 to be tracked and identified, including being associated with a particular patient and/or wearable. This embodiment also has at least one chemically-sensitive field-effect transistor (FET) 1136 that can be adapted for drug monitoring to detect chemical concentrations of a drug in the patient that may have been administered as part of the heart failure treatment plan, which can be used to monitor patient compliance with taking the prescribed drug. This embodiment also includes at least one reverse iontophoresis (RIP) module 1137, which can be used to detect one or more of a variety of analytes in the interstitial fluid. Analytes that can be monitored with the RIP module 1137 include ions such as sodium, potassium, calcium, magnesium, and phosphate, but can also include diuretics that may be prescribed to the patient.

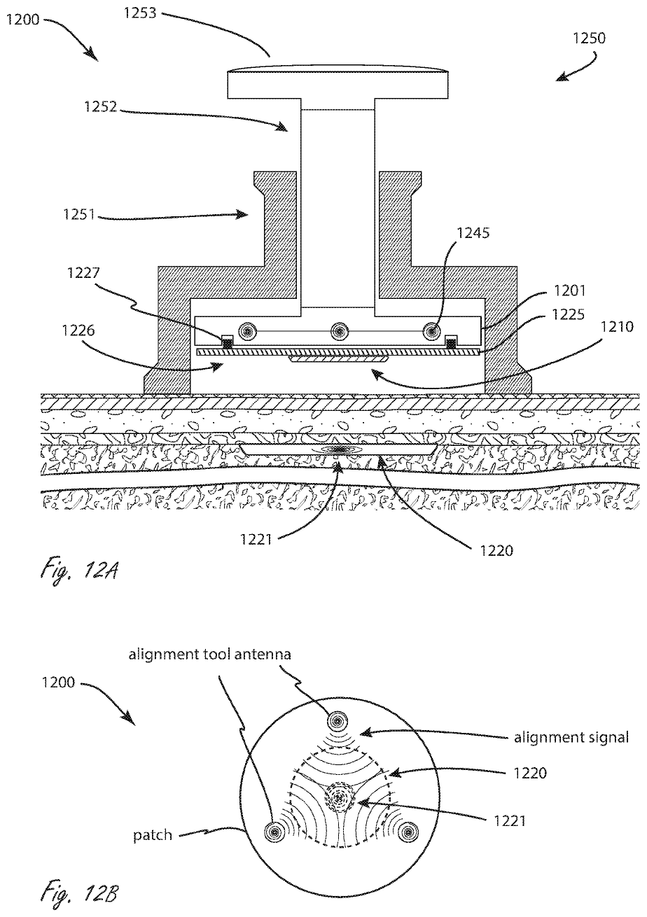

FIGS. 12A and 12B (side and top views, respectively) illustrate a system 1200 that includes an implantable portion 1220 with an antenna 1221. The system 1200 includes a wearable 1201 and a surface portion 1225 (with protruding optical emitter/receiver 1210 secured thereto), the two of which are stabilized via the alignment posts 1227 similar to that shown in FIGS. 8 and 9. The wearable 1201 and surface portion/patch 1225/1226 are shown ready to be accurately positioned on the patient in a desired location using an applicator/alignment tool 1250. The alignment tool 1250 includes an applicator housing 1251 with a first region sized to receive the wearable 1201 and patch 1226 therein, and a second region sized to receive the movable plunger 1252 therein. An actuator (not shown) is secured to the movable plunger 1252 to facilitate the downward movement of plunger 1252 via downward movement of the alignment display. The alignment tool 1250 can include an alignment display 1253 that can be transparent so that a user can visualize the wearable 1201. The wearable 1201 in this embodiment includes a plurality of alignment tool antennas 1245 (three shown), disposed around the peripheral region of the wearable 1201 and at 120 degrees to each other. When an electrical signal is communicated to the tool antennas 1245, an alignment signal is emitted from each of the alignment tools, and the alignment signals interact with each other. The system 1200 is adapted to emit an alert (audio, tactile (e.g., vibration), visual (e.g., lights), etc.) when the implant antenna 1221 is equidistant from the tool antennas 1245, which alerts the user that the wearable 1201 and patch 1226 are in a desired location to be applied to the patient. The user can then depress the plunger 1252 and apply the patch 1226 and wearable 1201 to the patient.

Any of the wearables herein can include one or more optical sensing systems. For example, the systems described herein can facilitate the use of photoplethysmography (“PPG”), which is a low-cost optical technique that can be used to detect blood volume changes in the microvascular bed of tissue based on expansion of capillaries due to the cardiac cycle. It is often used non-invasively to make measurements at the skin surface. The wearable can include one of more photoplethysmographic (PPG) sensors, which can operate in either transmissive mode and/or reflective mode. If in transmissive mode, a sensor is positioned to capture light transmitted from the light source (emitter) and through the tissue or interest (e.g., as with a pulse oximeter). One common location for PPG in reflective mode is the forehead. PPG can also be configured herein as multi-site photoplethysmography (MPPG), e.g., making simultaneous measurements from different locations, which allows gathering a wider array of information. In the PPG often one source is used, near IR, which is absorbed by the hemoglobin in the blood.

In some embodiments, optical sensing systems of the present technology can be utilized to measure various aspects of fluid status, including hypervolemia, hypovolemia, hemodilution, and/or hemoconcentration. These systems and measurements can be used as purely diagnostic indicators or as an input to titrate a drug or other modality in an appropriate therapy. For example, the systems may be used to monitor drug compliance for diuretics or other drugs which transiently shift the level of interstitial fluids.

Hypervolemia or fluid overload, increased and or increasing plasma volume, enlarged red blood cell width, and hemoconcentration have been associated with acute decompensation in heart failure. A primary tool in the treatment of decompensated heart failure is the administration of diuretics to reduce the level of hypervolemia and or plasma volume. An accurate measurement of hypervolemia, hypovolemia, hemodilution, and/or hemoconcentration can therefore be useful in assessing a heart failure patient's status as well as in determining accurate dosage for administration of diuretics. Likewise, because diuretics are used to treat other disorders, such as hypertension, the present technology can also be useful in assessing the efficacy of diuretics in disorders beyond heart failure.

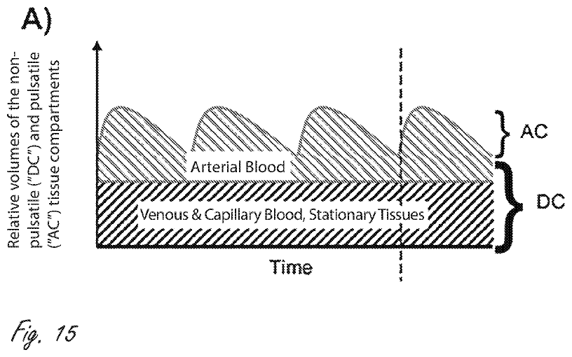

One way to measure the amount of fluid in tissue is to measure the absorbance of the tissue. The absorbance of tissue is based at least in part on the absorbance of the various components of the tissue, including, for example, dermal tissue, epidermal tissues, interstitial fluid, and/or blood (e.g., capillary, arterial, and/or venous). As best illustrated in FIG. 15, fluid measurement taken in tissues can be described as comprising multiple compartments. One model of use in this discussion comprises the following compartments: pulsatile (e.g., fluid volume that varies with time, such as arterial blood) or AC variation, and non-pulsatile (e.g., fluid volume that is generally static, such as venous, lymphatic, tissue interstitial fluids, cellular materials) or DC/background variation. The background will change slowly as a function of static blood and Hemoglobin (Hb) content.

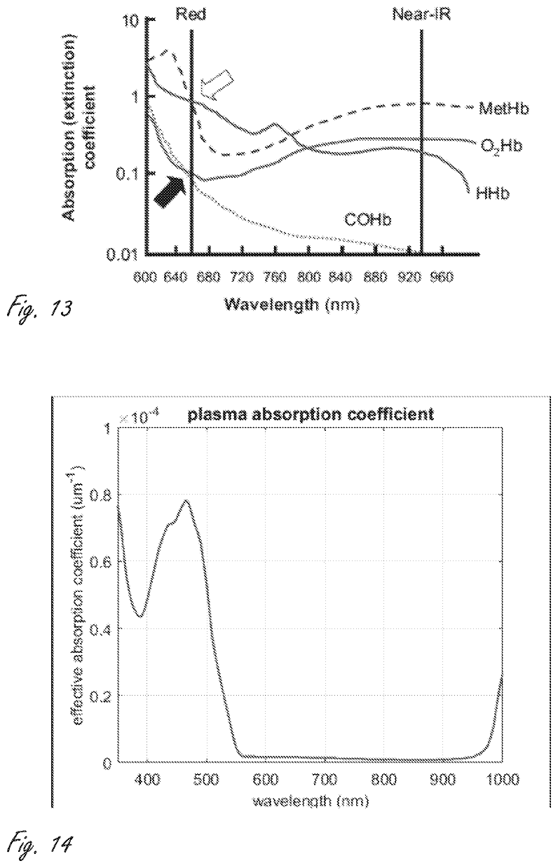

The absorbance of blood varies as a function of the state of Hemoglobin (Hb). FIG. 13, for example, is a graphical illustration of the absorbance of blood at various wavelengths for various states of Hemoglobin (Hb). As illustrated, the absorbance of the blood, in the range of 600 nm to 1200 nm, varies primarily as a function of whether Hb is oxygenated Hemoglobin (O₂Hb) or non-oxygenated Hemoglobin (HHb). Other forms of Hb, such as Carbon Monoxide bound Hb (COHb) and methylated Hb (MetHb) have other absorptive characteristics. Plasma has an absorbance peak around 480 nm. (FIG. 14).

Accordingly, the level of optical absorbance through a volume of tissue, in the passbands associated with Hb, will vary as a function of change in mass of Hb within the pathlength in the field of view, the time variance of the oxygenation state of the Hb within the path, and time variance in the pathlength across which the measurement is being made. The time varying aspects (e.g., oxygenation state of Hb and pathlength across which the measurement is being made) will change as a function of heart rate, respiration rate, and interstitial fluids building volume in the tissue (e.g., the tissue becoming edemic).

Accordingly, one aspect of monitoring decompensation in accordance with embodiments of the present technology is following the changes in optical absorbance of a volume of tissue. Over time, as the tissue becomes more edemic and the volume of Hb in the blood decreases, both the DC and AC absorbance levels will decrease when accounting for pathlength variation (e.g., increasing pathlength). Accordingly, decreasing absorbance levels may indicate an increased state of hypervolemia. Monitoring the absorbance near or above 800 nm, at which the HHb and O₂Hb display relatively equivalent absorbances, will minimize the effect of changes in pathlength, movement, and other variations not specific to the absorbance of the xxHb in the field of view on the measurement. In some embodiments, the saturation remains constant while the signal decreases.

Using a system having both an external component and an implanted component is expected to increase the efficiency, accuracy, and/or breadth of parameters that can be measured. For example, the external component can be configured for placement on the patient's skin and include a first sensor component (e.g., a light source, a reflector, etc.). The implanted component can be configured for placement beneath a dermal layer of skin and include a second sensor component (e.g., a light source, a reflector, etc.). Together, the first sensor component and the second sensor component can measure one or more physiologic parameters (e.g., absorbance) that indicate an amount of fluid in the tissue. In some embodiments, the external component may have a plurality of first sensor components to increase the accuracy and/or number of measurements that can be taken using the system. Accordingly, as provided above, in some embodiments, the present technology provides a system for measuring optical absorbance or other parameters that includes an implantable portion and a noninvasive portion.

In other embodiments, the tools are completely noninvasive. In some embodiments, the system measures, within the field of view, any combination of the ratio of Hemoglobin (Hb) to plasma which is used as a means of characterizing hemoglobin concentration. The system can be configured to measure the volume and or changes in the volume of plasma, the thickness or changes of thickness of the epidermis, and/or the relative conductivity of the epidermis, either parallel or normal to the surface or both, among other properties, changes in the proportion of xxHb in the various compartments (e.g., pulsatile vs. non-pulsatile, FIG. 15).

The systems can include the use of any or any combination of photo plethysmography (PPG), peripheral capillary oxygen saturation (SPO₂), multi wavelength SPO₂ (mSPO₂), electrical impedance tomography (EIT), and spectral EIT (EITs). The foregoing tools can generally detect optical signals centered in the neighborhood of, for example, 480 nm, 660 nm, 930 nm, etc. (e.g., wavelengths associated with various parameters). PPG, when used in a transmissive or in a reflective mode, relative to a volume of tissue, provides a volumetric measurement of the tissue contacted. SPO₂ provides a measure of the amount of O₂ saturated and unsaturated hemoglobin within an illuminated volume of tissue. mSPO₂ uses additional sources to differentiate hematocrit volume from plasma solids volume. EIT provides a measure of the electrical conductance of the volume of tissue between a set of measuring electrodes, wherein different tissues have differing electrical conductivities. The measured conductivity will be a function of the individual tissue conductivities in the path and the various path lengths and distributions of the various tissues. EITs provides a measure similar to EIT, but allow for differentiation between tissue within the electrical path as different tissues vary in conductivity as a function of frequency differently. It will be appreciated that any of the embodiments described herein can be configured to operate with EIT or sEIT in addition to or instead of optical means.

In some embodiments, the noninvasive portion of the system comprises a PPG/SPO₂ sensor. The noninvasive portion can comprise a PPG sensor alone used in a transmissive or reflective mode for the assessment of changes in the optical absorbance of blood to characterize the concentration of hematocrit in the blood. When using an SPO₂ sensor, volume changes can be differentiated relative to arterial vs venous filling. Respiratory changes can also be monitored.

In some embodiments, the noninvasive portion of the tool comprises a PPG/SPO₂ sensor in combination with EIT sensor. This combination allows for the measurement of volume by two methods each having differing responses to hematocrit vs plasma. Using these two methods allows for noise cancellation and supports differentiation of hematocrit from fluid volume.

In some embodiments, the invasive portion of the tool comprises one or more optical sources and at least one sensor affixed to a medical implant such that the sources illuminates the detector. The source(s) and sensor(s) can be arranged such that they can measure the absorbance of blood at one or more known wavelengths. The implant can be deployed within the vasculature. The optical implant can be used to measure the absorbance of the blood. The absorbance values or signals can be used to characterize the concentration of hematocrit and or hemoglobin in the blood.

In some embodiments, the tool is a hybrid tool comprising a source on the outside and a sensor on the inside with or without a means of measuring pathlength. The tool can comprise a source and a sensor on the outside and a reflector on the inside with or without a means of measuring pathlength. In some embodiments, the tool comprises one or more optical sources adjacent the skin, outside of the body and one or more sensors within the body. The sensor within the body can be mounted within a blood vessel. The sensors can be configured to gather power form an outside power source via any or any combination of RF, ultrasound (US), motion, acoustic sources. In some embodiments, the tool comprises one or more optical sources and one or more sensors affixed to the skin, outside of the body and an optically reflective device placed with in the body such that the energy form the source(s) is reflected back to the sensor(s). The tool can comprise a sensor mounted within a blood vessel. The sensor can be placed subcutaneously. The device can comprise a sensor for characterizing the distance between the reflector and the source(s) and sensor(s). In some embodiments, the system comprises a sensor configured to characterize an intensity value for the energy associated with a source on a detector. The distance characterization can be used to define a path length. In some embodiments, the distance characterization and intensity characterization can be used to characterize a concentration of Hb or hematocrit.

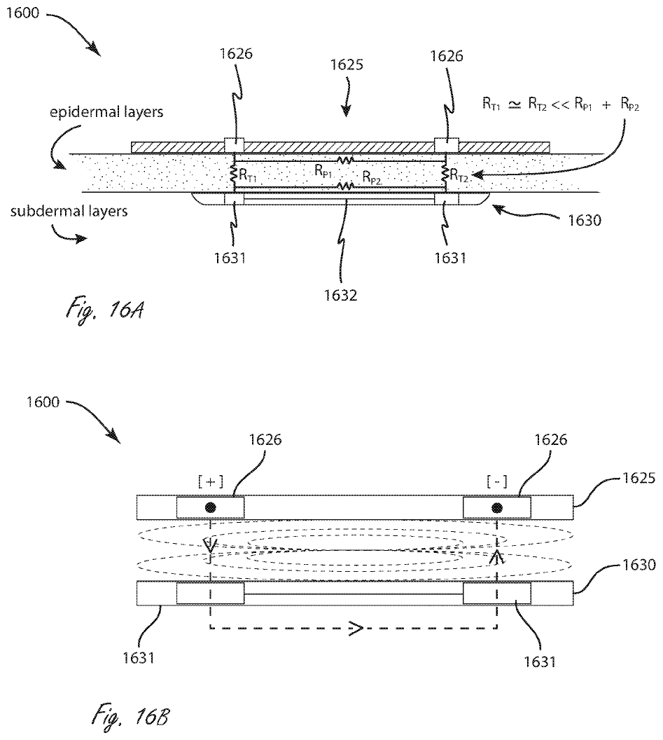

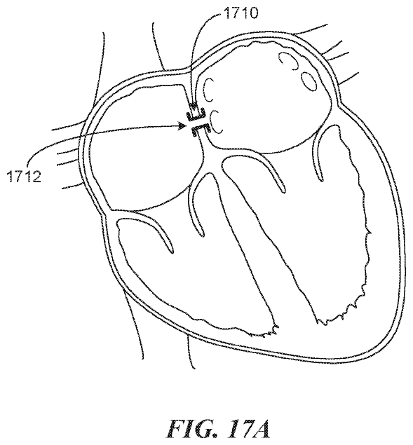

FIGS. 16A and 16B show another embodiment of a device comprising a wearable patch 1625 and an implanted portion 1630 that are configured to interact with one another. The wearable patch 1625 can include one or more electrodes 1626. Likewise, the implanted portion 1630 can include one or more electrodes 1631 within an insulated conductive element 1632. The one or more electrodes 1626 on the wearable patch 1625 can form an electrical circuit with, or otherwise communicate with, the one or more electrodes 1631, as best shown in FIG. 16B. By having electrodes on both the patch 1625 and the implanted portion 1630, additional measurements (e.g., electrical impedance R_{T1} and R_{T2}, FIG. 16A) can be taken.

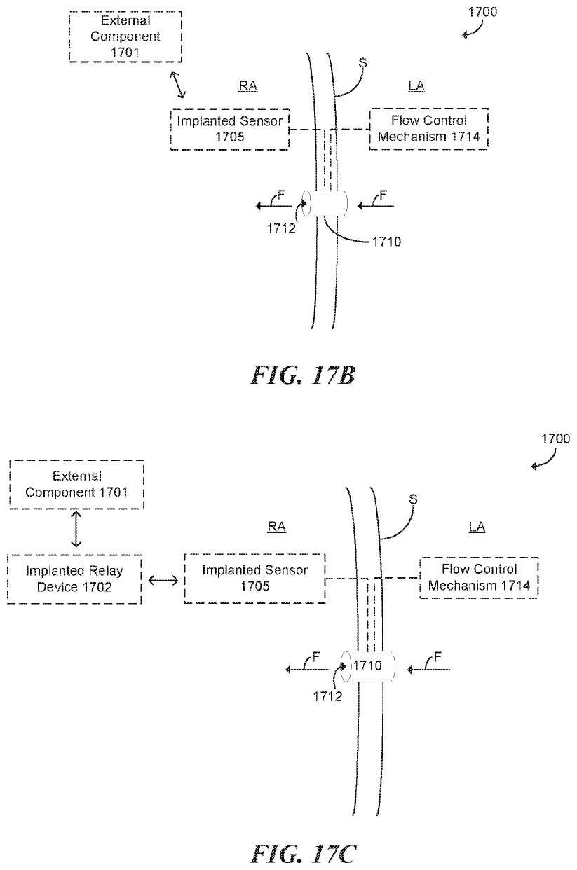

FIGS. 17A-17C illustrate an embodiment of a system 1700 for monitoring and/or treating heart failure in a patient. FIG. 17A is a schematic illustration of in implanted component 1710 for use with the system 1700. In the illustrated embodiment, the implanted component 1710 is an interatrial shunt device (the “device 1710”). The device 1710 can be implanted across a septal wall of a human heart such that a first end portion is in fluid communication with a left atrium and a second end portion is in fluid communication with a right atrium. The interatrial shunt device 1710 can have a lumen 1712 (FIG. 17B) extending between the first end portion and the second end portion to fluidly connect the left atrium and a right atrium. Without being bound by theory, shunting blood from the left atrium to the right atrium can provide an effective therapy in certain patients suffering from heart failure, such as those patients who have heart failure with preserved ejection fraction.

FIG. 17B is a schematic illustration of certain aspects of the system 1700. As provided above, the system 1700 includes the device 1710 having the lumen 1712 extending therethrough. The system 1700 can further include a flow control mechanism 1714 configured to change a size, shape, and/or other characteristic of the device 1710 to selectively modulate the flow of fluid through the lumen 1712. For example, the flow control mechanism 1714 can be configured to selectively increase a diameter of the lumen 1712 and/or selectively decrease a diameter of the lumen 1712 in response to an input. In other embodiments, the flow control mechanism 1714 is configured to otherwise affect a shape and/or geometry of the lumen 1712. Accordingly, the flow control mechanism 1714 can be operably coupled to the device 1710 and/or can be included within the device 1710 itself. In some embodiments, for example, the flow control mechanism 1714 is part of the device 1710 and at least partially defines the lumen 1712. In other embodiments, the flow control mechanism 1714 is spaced apart from but operably coupled to the device 1710.

The system 1700 can also include one or more implanted sensor(s) 1705. The one or more implanted sensor(s) 1705 can be configured to measure one or more parameters of the system 1700 (e.g., a characteristic or state of the device 1710 or lumen 1712) and/or one or more physiological parameters of the patient (e.g., left atrial pressure, right atrial pressure, etc.). The sensor(s) 1705 can be coupled (e.g., physically coupled) to the device 1710. For example, the sensor 1705 can be physically coupled to the interatrial shunt device 1710 (e.g., positioned on a left atrial or right atrial end portion of the device 1710, or positioned within a housing positioned across a portion of the septal wall S). In other embodiments, the sensor 1705 is not directly (e.g., physically) coupled to the device 1710, and can be positioned at a location within the heart spaced apart from the device 1710 (e.g., the left atrium LA, the right atrium RA, the septal wall S, etc.). For example, the system 1700 can include a first sensor positionable within or proximate to the left atrium LA to measure left atrial pressure, and a second sensor positionable within or proximate to the right atrium RA to measure right atrial pressure. Examples of sensor(s) 1705 suitable for use with the embodiments herein include, but are not limited to, pressure sensors, impedance sensors, accelerometers, force/strain sensors, proximity sensors, distance sensors, temperature sensors, flow sensors, optical sensors, cameras, microphones or other acoustic sensors, ultrasonic sensors, ECG or other cardiac rhythm sensors, SpO2 and other sensors adapted to measure tissue and/or blood gas levels, blood volume sensors, and other sensors known to those who are skilled in the art. In some embodiments, the system 1700 includes multiple different types of sensors, such as at least two, three, four, five, or more different sensors and, as noted previously, the sensors may be positioned at a variety of different locations within the patient.

The system 1700 can further include an external component 1701. The external component 1701 can be generally similar to any of the external components described herein, including, for example, a wearable device, a surface portion of an implantable device, a patch, or the like. In some embodiments the external component 1701 can include a power source configured to power the sensor 1705. In some embodiments, the external component 1701 and the implanted sensor 1705 can communicate (e.g., wirelessly communicate). For example, the external component 1701 can provide power to the sensor(s) 1705 and receive measurements from the sensor 1705. In some embodiments, the external component 1701 can instruct the implanted sensor(s) 1705 to record and transmit a physiological parameter. In such embodiments, the implanted sensor(s) 1705 may remain in a “sleep” mode until the external component 1701 directs (e.g., by sending a signal) the implanted sensor 1705 to take a reading. Without being bound by theory, taking on demand or periodic readings (e.g., as opposed to continuous readings) is expected to increase the lifespan of a battery included on the implanted sensor 1705.

FIG. 17C illustrates another embodiment of the system 1700. In particular, the system 1700 can include an implanted relay device 1702. The implanted relay device 1702 can be coupled to both the external device 1701 and the implanted sensor 1705 via, for example, a wireless or other connection. The implanted relay device 1702 can receive signals from the external component 1701 and transmit the received signals to the implanted sensor 1705. Likewise, the implanted relay device 1702 can receive signals from the implanted sensor 1705 and transmit the received signals to the external device 1701. The implanted relay device 1702 can be implanted in various locations, such as within the patient's vasculature and/or within a subcutaneous layer of skin to facilitate the connection between the external device 1701 and the implanted sensor 1705. In some embodiments, the relay device 1702 may be generally similar to those described in International Patent Application No. PCT/US2019/069106, filed Dec. 31, 2019, the disclosure of which is incorporated by reference herein in its entirety.

Any of the optical components and systems herein can include additional emitters at additional wavelengths and sensors configured to detect SpO₂ (peripheral capillary oxygen saturation), which is a known technique, such as in used in a pulse oximeter. Any of the implanted subcutaneous sensors herein may be used as part of SpO₂ detection (e.g., in transmissive mode). Additional emitters may be added to further distinguish plasma from hemoglobin. In SpO₂, typically two sources are used, one near IR and one visible (typically red). Both wavelengths are absorbed by hemoglobin with the near IR sensor being less sensitive to the oxygen saturation state of the hemoglobin and the visible wavelength being more sensitive to the oxygen saturation of the hemoglobin. In yet other embodiments an additional emitter may be used which is more sensitive to plasma then hematocrit.

In any of the embodiments herein the wearable can include one or more ultrasound assemblies that are adapted to provide distance measurements to implanted or tissue structures adjacent the wearable, which can be used to characterize or measure changes in thickness of the layers of skin, due to variations in the fluid volume associated with the cardiac and or respiratory cycle and or static fluid volume over longer periods of time. Changes in the thickness of one or more layers can be used to help determine changes in the optical pathlength for emitted light, which can delineate how much of a change in an electrical impedance measurement or an optical absorbance measurement results from a pathlength change vs an actual impedance or absorbance change respectively. Any of the wearables herein can include a motion detector (e.g., an accelerometer, gyroscope, or the like), which can help account for patient motion when taking other measurements (e.g., optical PPG readings). In any of the embodiments herein, the wearable can be any known wearable device, such as a watch, or a wrist-worn athletic/health device.

In any of the systems herein, an optical module can be used to detect fluorescence of a marker in the patient. For example, a fluorescent marker can be delivered into a patient from a surface portion of the system, the marker adapted to bind to a prescribed drug. A reverse iontophoresis module can be used to remove the drug from the patient, which should be labeled with the dye. The optical system can then detect the amount of drug (labeled) to determine if the patient is taking a prescribed drug.

The disclosure herein includes the following uses and functionality, some of which are described in more detail above. One or more motion devices (e.g., accelerometer and/or gyro) can be used to correct for motion artifact in sensed pressure. One or more motion devices (e.g., accelerometer and/or gyro) can be used to characterize activity of the patient. A patient's health can be characterized by characterizing changes in any of, including any combination thereof, blood pressure, SpO2, plethysmography in response to activity levels. Measurements obtained from any of the systems herein can be used to evaluate the efficacy of a drug titer. A drug dosage can be change by a physician in response to a change observed in any of, including any combination thereof, blood pressure, SpO2, plethysmographically sensed data, electrical impedance, etc., as measured by the device. A detected optical pathlength can be used to normalize readings from any of, including any combination thereof, plethysmographically sensed data, electrical impedance, SpO2, etc.

Any of the sensed and/or detected data herein can be used in combination with any other sensed data as a way of better cross correlating the detected data. This can lead to more accurate characterizations about the patient's condition, which may also help with compliance.

In some embodiments the wearables herein can be used as reusable components and the surface portions and/or implanted components can be considered disposables that are replaced when needed. The wearables can in this context house the relatively more expensive components.

The implantable components described herein can be temporary or permanent. For example, in some embodiments the implantable can be removable using known techniques. In some embodiments, aspects of the implantable components can be biodegradable. In some embodiments, the implantable components are configured to remain implanted for a prolonged period of time (e.g., at least one month, at least three months, at least one year, etc.).

### Examples

Several aspects of the present technology are set forth in the following examples:

1. A patient monitoring system, comprising:
an implantable portion sized and configured to be implanted within a subject, optionally sized and configured to be placed in a blood vessel of the subject, the implantable portion including a sensor, such as a pressure and/or optical sensor.

2. The system of example 1, wherein the implantable portion includes a first portion configured to be positioned in a blood vessel of the subject, and a second portion configured to be placed outside of the blood vessel, the first portion coupled to the second portion.

3. A system of any preceding example, wherein the implantable portion includes a reflector and/or sensor.

4. A system of example 3, wherein the reflector and/or sensor is physically coupled to a portion of the implantable portion disposed in a blood vessel.

5. A system of example 3, wherein the reflector and/or sensor is not physically coupled to a portion of the implantable portion disposed in a blood vessel.

6. A system of any preceding example, further comprising a surface portion (e.g.; in the form of an adhesive patch) configured to be adhered to a surface of the patient.

7. A system of any preceding example, wherein the implanted portion is physically connected to the surface portion.

8. A system of any preceding example, wherein the implanted portion is not physically connected to the surface portion.

9. A system of example 8, wherein the implanted portion is configured to be in communication with the surface portion.

10. A system of any preceding example, further comprising a wearable device configured to be worn by the subject.

11. A system of any preceding example, wherein a wearable is configured to interface with a surface portion of the system.

12. A system of any preceding example, wherein a wearable comprises one or more sensors (e g, pressure, motion, optical, impedance).

13. A system of any preceding example, wherein a wearable comprises one or more optical sensors adapted to emit light towards and through the skin of the subject.

14. A system of any preceding example, wherein a wearable includes an interface feature configured to interface with a surface portion interface feature to increase the stability of the wearable relative to the surface portion.

15. A system of any preceding example, wherein a skin portion has a wearable optical interface feature (e.g., a window) that is configured to interface with an optical emitter and/or sensor of the wearable to position the wearable in a desired position relative to the skin portion, optionally wherein the wearable optical interface feature provides for the wearable to directly access the skin of the patient.

16. A system of any preceding example, wherein the implanted portion is physically coupled to a surface portion, but not physically coupled to a reflector and/or sensor.

17. A system of any preceding example, wherein a reflector and/or sensor is configured to be implanted subcutaneously.

18. A system of any preceding examples, wherein a wearable and a implanted reflector and/or sensor are configured to allow light to pass through the subject from an emitter to a sensor to sense data indicative of one or more patient parameters (e.g., SpO2).

19. A system that includes one or more feature of the alignment device in FIGS. 12A and 12B, including methods of use to position a surface portion and/or wearable on a subject.

20. Any of the wearable devices herein, including any and all methods of use.

21. An intra-arterial blood pressure system, comprising a patient interface and a monitor, at least a portion of the patient interface sized to be disposed in an artery, such as a radial artery.

22. The system of example 21 wherein the patient interface includes a pressure transducer, optional adapted to be inside the artery or disposed in an external component outside the artery.

23. The system of any example herein wherein the monitor is a component in wireless communication with the patient interface.

24. The system of any example herein wherein the patient interface includes an indwelling portion and an external portion.

25. The system of any example herein wherein the patient interface includes an indwelling portion, which is adapted to be reversibly secured to a stiffening component to stiffen at least a portion of the indwelling portion during delivery, and cause the indwelling portion to be less stiff after its removal.

26. The system of any example herein wherein a pressure transducer is disposed at a distal region, optionally a distal end, of an access device.

27. The system of any example herein wherein a stiffening component is an elongate device, such as an introducer stylet.

28. The system of any example herein wherein the distal end of an access device is sharped, to allow it to be pierced through a patient's skin.

29. The system of any example herein wherein an access device of a patient interface includes a pressure capturing zone, adapted to transmit blood pressure to a pressure transducer.

30. The system of any example herein wherein a pressure capture zone includes a relatively thin walled portion of an access device.

31. The system of any example herein wherein a pressure capture zone includes a fluid or gel therein.

32. The system of any example herein further including a removable introducer stylet.

33. The system of any example herein wherein an external portion of a patient interface includes system conditioning components.

34. A patient treatment system for treating heart failure in a patient, the system comprising:
a shunt having a lumen extending therethrough, wherein, when the shunt is implanted in the patient, the lumen is configured to fluidly couple a left atrium and a right atrium of the patient;
a sensor implantable into the patient and operably coupled to the shunt, wherein the sensor is configured to measure one or more parameters corresponding to a physiological parameter of the patient and/or a characteristic of the shunt; and
an external component wirelessly coupled to the sensor, wherein the external component is configured to be worn by or otherwise adhered to the patient.

35. The patient treatment system of example 34 wherein the sensor is configured to measure a physiological parameter of the patient.

36. The patient treatment system of example 35 wherein the physiological parameter is blood pressure.

37. The patient treatment system of any of examples 34-36 wherein the sensor is configured to measure a characteristic of the shunt.

38. The patient treatment system of any of examples 34-37, further comprising a flow control element configured to change a shape and/or size of the lumen.

39. The patient treatment system of example 38 wherein the flow control element is configured to change the shape and/or size of the lumen based at least in part on a sensed physiologic parameter.

40. The patient treatment system of any of examples 34-39 wherein, in operation and in response to a user input, the external component can direct the sensor to record a measurement of the parameter.

41. The patient treatment system of any of examples 34-40 wherein, in operation and in response to a user input, the external component can direct the sensor to transmit a recorded measurement of the parameter to the external component or device positioned external to the patient.

42. The patient treatment system of any of examples 34-41 wherein the external component is an adhesive patch.

43. A patient treatment system for treating heart failure in a patient, the system comprising:
a shunt having a lumen extending therethrough, wherein, when the shunt is implanted in the patient, the lumen is configured to fluidly couple a left atrium and a right atrium of the patient;
a sensor implantable into the patient and in communication with the shunt, wherein the sensor is configured to measure one or more parameters;
an external component configured to be worn or otherwise adhered to the patient; and
an implantable relay device operably coupled to the sensor and the external component, wherein the implantable relay device is configured to (i) receive a first signal from the external component and transmit a second signal corresponding to the first signal to the sensor, and (ii) receive a third signal from the sensor and transmit a fourth signal corresponding to the third signal to the external component.

44. The patient treatment system of example 43 wherein the first signal includes an instruction for the sensor to record a measurement of the parameter.

45. The patient treatment system of examples 43 or 44 wherein the third signal includes a recorded measurement of the parameter.

46. The patient treatment system of any of examples 43-45 wherein the implantable relay device is physically connected to the external component.

47. The patient treatment system of any of examples 43-45 wherein the implantable relay device is wirelessly coupled to the external component.

48. The patient treatment system of any of examples 43-47 wherein the implantable relay device is physically connected to the sensor.

49. The patient treatment system of any of examples 43-47 wherein the implantable relay device is wirelessly coupled to the sensor.

50. A patient monitoring system, comprising:
an implantable device, wherein the implantable device includes—
   an implantable portion sized and configured to be implanted within a patient, the implantable portion including a sensor configured to measure one or more physiological parameters, and
   a surface portion coupled to the implantable portion and configured to be adhered to a surface of the patient; and
a wearable device configured to engage the surface portion to communicate with the implantable portion.

51. The system of example 50 wherein the implantable portion includes a first portion configured to be positioned in a blood vessel of the patient, and a second portion configured to be placed outside of the blood vessel, the first portion coupled to the second portion.

52. The system of examples 50 or 51 wherein the implantable portion includes a reflector and/or sensor.

53. The system of example 52 wherein the reflector and/or sensor is physically coupled to a portion of the implantable portion disposed in a blood vessel.

54. The system of example 52 wherein the reflector and/or sensor is not physically coupled to a portion of the implantable portion disposed in a blood vessel.

55. The system of any of examples 50-54 wherein the implantable portion is physically connected to the surface portion.

56. The system of any of examples 50-55 wherein the implantable portion is not physically connected to the surface portion.

57. The system of any of examples 50-56 wherein the wearable device includes one or more sensors.

58. The system of any of examples 50-57 wherein the wearable device includes one or more optical sensors adapted to emit light towards and through the skin of the patient.

59. The system of any of examples 50-58 wherein the wearable device includes a first interface feature and the surface portion includes a second interface feature, and wherein the first interface feature is configured to interface with the second interface feature increase the stability of the wearable device relative to the surface portion.

60. A system for monitoring patient status, the system comprising:
an external component configured for placement on a patient's skin, wherein the external component includes a first sensor component; and
an implantable component configured for placement beneath a dermal layer of skin, wherein the implantable component includes a second sensor component,
wherein the first and second sensor components are configured to measure a physiological parameter indicative of an amount of fluid in the tissue.

61. The system of example 60 wherein the first sensor component is a light source.

62. The system of example 60 wherein the first sensor component is a reflector.

63. The system of any of examples 60-62 wherein the second sensor component is a light source.

64. The system of any of examples 60-62 wherein the second sensor component is a reflector.

65. The system of any of examples 60-64 wherein the external component includes a plurality of first sensor components.

66. The system of any of examples 60-65 wherein the physiologic parameter is tissue absorbance.

### CONCLUSION

As used herein, the terms “interatrial device,” “interatrial shunt device,” “IAD,” “IASD,” “interatrial shunt,” and “shunt” are used interchangeably to refer to a device that, in at least one configuration, includes a shunting element that provides a blood flow between a first region (e.g., a LA of a heart) and a second region (e.g., a RA or coronary sinus of the heart) of a patient. Although described in terms of a shunt between the atria, namely the left and right atria, one will appreciate that the technology may be applied equally to devices positioned between other chambers and passages of the heart, or between other parts of the cardiovascular system. For example, any of the shunts described herein, including those referred to as “interatrial,” may be nevertheless used and/or modified to shunt between the LA and the coronary sinus, or between the right pulmonary vein and the superior vena cava. Moreover, while the disclosure herein primarily describes shunting blood from the LA to the RA, the present technology can be readily adapted to shunt blood from the RA to the LA to treat certain conditions, such as pulmonary hypertension. For example, mirror images of embodiments used to shunt blood from the LA to the RA can be used to shunt blood from the RA to the LA.

As described above, embodiments of the present disclosure may include some or all of the following components: a battery, supercapacitor, or other suitable power source; a microcontroller, FPGA, ASIC, or other programmable component or system capable of storing and executing software and/or firmware that drives operation of an implant; memory such as RAM or ROM to store data and/or software/firmware associated with an implant and/or its operation; wireless communication hardware such as an antenna system configured to transmit via Bluetooth, WiFi, or other protocols known in the art; energy harvesting means, for example a coil or antenna that is capable of receiving and/or reading an externally-provided signal which may be used to power the device, charge a battery, initiate a reading from a sensor, or for other purposes. Embodiments may include portions that are radiopaque and/or ultrasonically reflective to facilitate image-guided implantation or image guided procedures using techniques such as fluoroscopy, ultrasonography, or other imaging methods. Embodiments of the system may include specialized delivery catheters/systems that are adapted to deliver an implant and/or carry out a procedure. Systems may include components such as guidewires, sheaths, dilators, and multiple delivery catheters. Components may be exchanged via over-the-wire, rapid exchange, combination, or other approaches.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments. For example, although this disclosure has been written to describe apparatuses implanted within certain parts of the body, it should be appreciated that similar embodiments could be utilized for apparatuses implanted in or positioned at a variety of other regions of the body.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A patient treatment system for treating heart failure in a patient, the system comprising:
a shunt having a lumen extending therethrough, wherein, when the shunt is implanted in the patient, the lumen is configured to fluidly couple a left atrium and a right atrium of the patient;
a sensor implantable into the patient and operably coupled to the shunt, wherein the sensor is configured to measure one or more parameters corresponding to a physiological parameter of the patient and/or a characteristic of the shunt; and
an external component wirelessly coupled to the sensor, wherein the external component is configured to be worn by or otherwise adhered to the patient.

2. The patient treatment system of claim 1 wherein the sensor is configured to measure a physiological parameter of the patient.

3. The patient treatment system of claim 2 wherein the physiological parameter is blood pressure.

4. The patient treatment system of claim 1 wherein the sensor is configured to measure a characteristic of the shunt.

5. The patient treatment system of claim 1; further comprising a flow control element configured to change a shape and/or size of the lumen.

6. The patient treatment system of claim 5 wherein the flow control element is configured to change the shape and/or size of the lumen based, at least in part, on the sensed physiologic parameter.

7. The patient treatment system of claim 1 wherein, in operation and in response to a user input, the external component can direct the sensor to record a measurement of the parameter.

8. The patient treatment system of claim 1 wherein, in operation and in response to a user input, the external component can direct the sensor to transmit a recorded measurement of the parameter to the external component or device positioned external to the patient.

9. The patient treatment system of claim 1 wherein the external component is an adhesive patch.

10. A patient treatment system for treating heart failure in a patient, the system comprising:
a shunt having a lumen extending therethrough, wherein, when the shunt is implanted in the patient, the lumen is configured to fluidly couple a left atrium and a right atrium of the patient;
a sensor implantable into the patient and in communication with the shunt, wherein the sensor is configured to measure one or more parameters;
an external component configured to be worn or otherwise adhered to the patient; and
an implantable relay device operably coupled to the sensor and the external component, wherein the implantable relay device is configured to (i) receive a first signal from the external component and transmit a second signal corresponding to the first signal to the sensor, and (ii) receive a third signal from the sensor and transmit a fourth signal corresponding to the third signal to the external component.

11. The patient treatment system of claim 10 wherein the first signal includes an instruction for the sensor to record a measurement of the parameter.

12. The patient treatment system of claim 10 wherein the third signal includes a recorded measurement of the parameter.

13. The patient treatment system of claim 10 wherein the implantable relay device is physically connected to the external component.

14. The patient treatment system of claim 10 wherein the implantable relay device is wirelessly coupled to the external component.

15. The patient treatment system of claim 10 wherein the implantable relay device is physically connected to the sensor.

16. The patient treatment system of claim 10 wherein the implantable relay device is wirelessly coupled to the sensor.

17. A patient monitoring system, comprising:
an implantable device, wherein the implantable device includes—
an implantable portion sized and configured to be implanted within a patient, the implantable portion including a sensor configured to measure one or more physiological parameters, and
a surface portion coupled to the implantable portion and configured to be adhered to a surface of the patient; and
a wearable device configured to engage the surface portion to communicate with the implantable portion.

18. The system of claim 17 wherein the implantable portion includes a first portion configured to be positioned in a blood vessel of the patient, and a second portion configured to be placed outside of the blood vessel, the first portion coupled to the second portion.

19. The system of claim 17 wherein the implantable portion includes a reflector and/or sensor.

20. The system of claim 19 wherein the reflector and/or sensor is physically coupled to a portion of the implantable portion disposed in a blood vessel.

21. The system of claim 19 wherein the reflector and/or sensor is not physically coupled to a portion of the implantable portion disposed in a blood vessel.

22. The system of claim 17 wherein the implantable portion is physically connected to the surface portion.

23. The system of claim 17 wherein the implantable portion is not physically connected to the surface portion.

24. The system of claim 17 wherein the wearable device includes one or more sensors.

25. The system of claim 17 wherein the wearable device includes one or more optical sensors adapted to emit light towards and through the skin of the patient.

26. The system of claim 17 wherein the wearable device includes a first interface feature and the surface portion includes a second interface feature, and wherein the first interface feature is configured to interface with the second interface feature increase the stability of the wearable device relative to the surface portion.

27. A system for monitoring patient status, the system comprising:
an external component configured for placement on a patient's skin, wherein the external component includes a first sensor component; and
an implantable component configured for placement beneath a dermal layer of skin, wherein the implantable component includes a second sensor component,
wherein the first and second sensor components are configured to measure a physiological parameter indicative of an amount of fluid in the tissue.

28. The system of claim 27 wherein the first sensor component is a light source.

29. The system of claim 27 wherein the first sensor component is a reflector.

28. The system of claim 27 wherein the second sensor component is a light source.

29. The system of claim 27 wherein the second sensor component is a reflector.

30. The system of claim 27 wherein the external component includes a plurality of first sensor components.

31. The system of claim 27 wherein the physiologic parameter is tissue absorbance.
